(19) United States
(12) Patent Application Publication
(10) Pub. No.: US 2025/0170049 A1
(43) Pub. Date: May 29, 2025

(54) Flavored Perfume

(71) Applicant: Sweetums, Inc., Carlsbad, CA (US)

(72) Inventor: Alisa Brady, Carlsbad, CA (US)

(21) Appl. No.: 18/961,474

(22) Filed: Nov. 27, 2024

Related U.S. Application Data
(60) Provisional application No. 63/604,169, filed on Nov. 29, 2023.

(51) Int. Cl. A61K 8/92; A23L 27/10; A61Q 1/06; A61Q 13/00

(52) U.S. Cl. CPC A61K 8/922; A23L 27/10; A61K 8/927; A61Q 1/06; A61Q 13/00

(57) ABSTRACT

A flavored perfume is disclosed that includes both a scent component and a taste component, enabling users and others to experience both a smell and a taste of the product. The flavored perfume includes a carrier, a scent component, and a flavoring component, and is intended for external use on the body.

### CROSS REFERENCE TO RELATED APPLICATIONS

This application claims priority on U.S. Provisional Patent Application No. 63/604,169 entitled “Flavored Perfume”, filed on Nov. 29, 2023, which is incorporated by reference herein in its entirety and for all purposes.

### STATEMENT REGARDING FEDERALLY SPONSORED RESEARCH OR DEVELOPMENT

This invention was not federally sponsored.

### FIELD OF THE INVENTION

This invention relates to the general field of perfumes, and, more specifically, to a flavored perfume product that is applied in a stick or spray formulation.

### BACKGROUND OF THE INVENTION

The use of scents for pleasurable interactions has a rich history spanning thousands of years. The practice dates back approximately 4,000 years to the Mesopotamian culture, where incense was first created. Around 3,000 years ago, the ancient Egyptians began burning resins and special woods as part of religious ceremonies. They later developed perfumes, which were used to scent baths, and scented oils were applied to their bodies.

Liquid perfumes, as we know them today, emerged several thousand years later in ancient Greece. Hippocrates is said to have prescribed perfumes for “fumigation” and disease prevention. Theophrastus, known as the father of botany, authored a book on fragrances and the plants that could be used to create different scents. Many animal-based scents were also utilized. Shakespeare even wrote of Cleopatra using perfumes to seduce Marc Antony.

As distillation processes advanced, the extraction of perfumes became more refined, commercialized, and widespread. While perfumes created before were relatively simple and often derived from a single flower species, by the 19th century, perfume formulation became significantly more complex. Eau de cologne, a forerunner to modern cologne, was invented by an Italian barber in the city of Cologne or Koln, Germany. Today, the perfume market in the United States alone exceeds six billion dollars annually.

The use of flavorings in food has an even older origin. Although it is impossible to pinpoint the first individual who discovered the practice, the use of flavorings likely began tens of thousands of years ago. By the height of the Egyptian Empire, various herbs were used to flavor food, including sesame, thyme, cinnamon, coriander, dill, cumin, and marjoram. During the Middle Ages, herbs and spices were not only used to enhance flavor but also to preserve food.

Despite the long history of perfumes and flavorings, their paths of development have largely been parallel rather than interconnected. To date, the concept of a “tastable” perfume—one that combines both scent and flavor—has not been realized.

Several existing patents describe technologies related to solid perfumes, but none address the concept of a flavored perfume as described in this application. For instance:

Korean Patent Publication No. KR20190050186A describes a solid perfume composition including a phytoncide scent, intended to relieve insomnia and stress. While the composition is noted for its convenience, it does not include any flavor components.

Chinese Patent Publication No. CN109700699A discloses a solid-state perfume prepared from a carrier mixture, which may include various oils, waxes, and essential oils. The product is designed for long-lasting fragrance with minimal volatility but, like other prior art, does not incorporate a taste component.

Chinese Patent Publication No. CN103330657A discusses a method for creating a solid perfume stick, involving a specific mixture of oils, waxes, and fragrances. Again, there is no mention of incorporating flavor into the product.

Chinese Patent Publication No. CN115212139A covers a rod-shaped cosmetic balm that provides a lasting fragrance. The balm's primary function is to maintain fragrance over time, with no reference to any flavor component.

U.S. Pat. No. 11,484,616B2 describes a solid fragrance composition featuring porous particles for improved fragrance persistence. This invention focuses solely on fragrance retention and does not include any flavoring elements.

U.S. Pat. No. 8,425,886B2 pertains to a deodorant formulation for improved efficacy and stability. While it describes solid formulations, it is directed at odor control and does not address flavored perfumes.

Despite significant advancements in both the perfume and food flavoring industries, no prior art provides a solution for a flavored perfume. Thus, there has existed a long-felt need for an innovative product that combines both scent and taste. The current invention aims to fill this gap by providing a flavored perfume that can be applied both as a stick or spray.

### SUMMARY OF THE INVENTION

The present invention provides a flavored perfume product comprising both a scent component and a taste component, allowing the user and others to enjoy both the fragrance and the flavor of the product. The product is formulated to provide both a smell and a taste, including a carrier, a scent component, and a flavoring component. It is intended for external use on a user's body and can be applied either as a stick or a spray.

One aspect of the present invention, the stick version of the product includes a carrier selected from a wax-based formulation, which may include ingredients such as coconut oil, jojoba oil, vitamin E, shea butter, and beeswax. These ingredients not only serve as the base for the product but also provide health benefits to the skin. The flavoring component, such as stevia, is incorporated into the formulation, allowing users to experience both the scent and the taste of the product.

Another aspect of the present invention, the spray version of the product includes a carrier selected from oil-based liquids and water-based liquids. The formulation also includes scent components and flavoring components, offering the users experience to experience of both smelling and tasting the product.

In yet another aspect of the present invention, a perfume is combined with a flavor to create a flavored perfume. It is contemplated that both oil-based and water-based carriers can be used.

The goal of this product is to provide a flavored perfume product that can be applied either by rolling on or spraying, allowing the user or another person to both smell and taste the substance by licking it.

It is therefore an object of the present invention to provide a product that combines both a scent and a flavor.

Another object of the invention is to provide a product in a compact, portable container for ease of carrying by the user.

A further object of the invention is to offer a product in both solid (stick) and liquid (spray) forms.

A final object of the invention is to provide a product that can be both smelled by the nose and tasted by the tongue.

There has thus been outlined, rather broadly, the more important features of the invention in order that the detailed description thereof may be better understood, and in order that the present contribution to the art may be better appreciated. There are additional features of the invention that will be described hereinafter, and which will form the subject matter of the claims appended hereto. The features listed herein, and other features, aspects and advantages of the present invention will become better understood with reference to the following description and appended claims. The accompanying drawings, which are incorporated in and constitute part of this specification, illustrate embodiments of the invention and, together with the description, serve to explain the principles of the invention.

### BRIEF DESCRIPTION OF THE DRAWINGS

One preferred form of the invention will now be described with reference to the accompanying drawings.

FIG. 1 illustrates a device incorporating a product of the present invention.

### DETAILED DESCRIPTION OF THE INVENTION

The present invention will now be described more fully hereinafter with reference to the accompanying drawings in which a preferred embodiment of the invention is shown. This invention may, however, be embodied in many different forms and should not be construed as being limited to the embodiment set forth herein. Rather, the embodiment is provided so that this disclosure will be thorough, and will fully convey the scope of the invention to those skilled in the art.

The following terms and phrases have the meanings indicated below, unless otherwise provided herein. This disclosure may employ other terms and phrases not expressly defined herein. Such other terms and phrases shall have the meanings they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly indicates otherwise. For example, reference to “a product” encompasses a single product as well as two or more product, and the like.

As used herein, “for example,” “for instance.” “such as,” or “including” are meant to introduce examples that further clarify more general subject matter. Unless otherwise expressly indicated, such examples are provided only to aid in understanding embodiments illustrated in the present disclosure and are not meant to be limiting in any fashion. Nor do these phrases indicate any kind of preference for the disclosed embodiment.

As used herein “flavoring or taste component” is meant to refer to a compound, which is used in flavoring or taste preparations or compositions to impart a hedonic effect. In other words, such an ingredient, to be considered as being a flavoring or taste, must be recognized by a person skilled in the art as being able to impart or modify in a positive or pleasant way the flavor or the taste a composition, and not just as having a flavor or a taste.

As used herein “perfuming or scenting or fragrance co-ingredient” is meant to refer to a compound, which is used in perfuming or scenting or fragrance preparations or compositions to impart a hedonic effect. In other words, such an ingredient, to be considered as being a perfuming or scenting or fragrance one, must be recognized by a person skilled in the art as being able to impart or modify in a positive or pleasant way the smell or the odor of a composition, and not just as having a smell or an odor.

In one embodiment of the invention, the flavored perfume comprising both a smell component and a taste component. In an exemplary embodiment, the invention combines a perfume with a flavor, to create a flavored perfume. The product is formulated to provide both a smell and a taste, and it includes a carrier, scent components and flavoring compounds. The product is intended for external use on a user's body and can be applied both as a stick or a spray.

In one embodiment, the stick version of the product includes a carrier selected from a wax-based formulation, which may include ingredients such as coconut oil, jojoba oil, vitamin E, shea butter, and beeswax. These ingredients not only serve as the base for the product but also provide health benefits to the skin. The flavoring components, such as stevia, is incorporated into the formulation, allowing users to experience both the scent and the taste of the product.

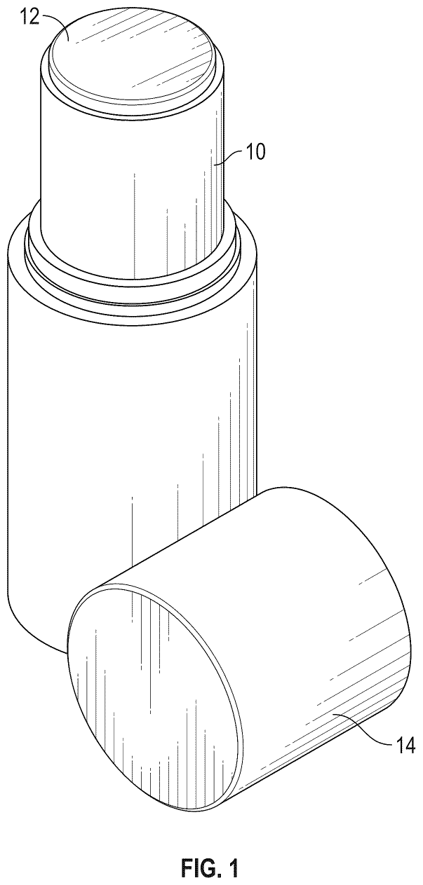

For example, according to one embodiment of the present invention, the stick version of the product is illustrated in FIG. 1, where a device 10 incorporates the product 12, which is covered by cover 14 when not in use. In a preferred embodiment, the product is a lipstick, balm, perfume, roll-on perfume, or any similar product without limiting the scope of the invention.

The stick version of the product is formulated using a wax-like base that can be easily applied to the skin, allowing for a controlled release of scent and flavor. The primary ingredient of the stick version is a carrier portion that is selected from a group of natural, skin-benefiting ingredients. These ingredients are specifically chosen for their health-promoting properties for the skin, as well as their ability to provide a smooth, stable base for the scent and flavor to be incorporated into the product.

In another embodiment, the spray version of the product includes a carrier selected from oil-based liquids and water-based liquids. The formulation also includes scent components and flavoring components, offering the user a similar dual-sensory experience of both smelling and tasting the product.

The purpose of the present invention is to allow the user, as well as others around the user, to experience both the smell and the taste of the product through simple external application to the body.

The carrier portion selected from a wax-based formulation not only contribute to the desired texture and consistency of the product but also offer various health benefits to the skin. For example, coconut oil is known for its moisturizing properties, jojoba oil for its skin-healing and anti-inflammatory effects, vitamin E for its antioxidant properties, shea butter for its skin-softening benefits, and beeswax for its ability to create a protective barrier on the skin.

In addition to the carrier portion, the product includes a scent portion that is carefully selected to provide a pleasing fragrance. The scent is incorporated into the wax-like base during the manufacturing process, ensuring that the fragrance is evenly distributed and long-lasting on the skin. The scent portion may consist of natural or synthetic essential oils, fragrances, or a combination thereof, designed to give the user a pleasant aromatic experience.

The flavoring portion of the product is derived from a natural ingredient such as stevia, a non-caloric, naturally sweet substance. The flavoring portion is included to provide a unique and novel experience, where the user not only enjoys the fragrance of the product but also tastes a subtle sweetness when they lick the applied area. This combination of flavor and scent enhances the sensory experience, making it a truly multi-sensory flavored perfume product.

The flavoring portion provides a mild, pleasant taste when the product is licked. The stevia is used as a natural flavoring agent, adding a sweet taste without the need for sugar or artificial sweeteners.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

What is claimed is:

1. A flavored perfume product providing both a smell and a taste, consisting of:
a carrier portion;
a scent portion configured to provide a smell component; and
a flavor portion configured to provide a taste component.

2. The flavored perfume product of claim 1, wherein the carrier portion is at least selected from a group consisting of coconut oil, jojoba oil, vitamin E, shea butter, and beeswax.

3. The flavored perfume product of claim 1, wherein the flavor portion is stevia, wherein the flavored perfume has a product, and where the product is selected from the group consisting of is lipstick, balm, perfume, and roll-on perfume.

4. The flavored perfume product of claim 1, wherein the flavored perfume product is a solid.

5. The flavored perfume product of claim 1, wherein the flavored perfume product is rolled onto a user's body in a manner that allows the user and others to both smell the flavored perfume product with their nose and taste the flavored perfume product with their tongue.

6. The flavored perfume product of claim 1, wherein the carrier portion is selected from a group consisting of oil-based liquids and water-based liquids.

7. The flavored perfume product of claim 1, wherein the flavored perfume product is a liquid, wherein the flavored perfume product is sprayed onto a user's body in a manner that allows the user and others to both smell the flavored perfume product with their nose and taste the flavored perfume product with their tongue.

8. The flavored perfume product of claim 1, wherein the carrier portion is selected from a group consisting of coconut oil, jojoba oil, vitamin E, shea butter, and beeswax, wherein the flavor portion is stevia, wherein the flavored perfume product is a solid, wherein the flavored perfume product is rolled onto a user's body in a manner that allows the user and others to both smell the flavored perfume product with their nose and taste the flavored perfume product with their tongue.

9. A flavored perfume providing both a smell and a taste, comprising of: a carrier portion; a scent portion; and a flavor portion.

10. A flavored perfume of claim 9, wherein the carrier portion is at least selected from a group consisting of coconut oil, jojoba oil, vitamin E, shea butter, and beeswax.

11. The flavored perfume of claim 9, wherein the flavor portion is stevia, wherein the flavored perfume has a product, and where the product is selected from the group consisting of is lipstick, balm, perfume, and roll-on perfume.

12. The flavored perfume of claim 9, wherein the flavored perfume is a solid.

13. The flavored perfume of claim 9, wherein the flavored perfume is rolled onto a user's body in a manner that allows the user and others to both smell the flavored perfume with their nose and taste the flavored perfume with their tongue.

14. The flavored perfume of claim 9, wherein the carrier portion is selected from a group consisting of oil-based liquids and water-based liquids.

15. The flavored perfume of claim 9, wherein the flavored perfume is a liquid.

16. The flavored perfume of claim 9, wherein the flavored perfume is sprayed onto a user's body in a manner that allows the user and others to both smell the flavored perfume with their nose and taste the flavored perfume with their tongue.

17. A flavored perfume for external use on a user's body, providing both a smell and a taste, comprising:
a carrier portion is at least selected from a group comprising of coconut oil, jojoba oil, vitamin E, shea butter, and beeswax, or at least from a group comprising of oil-based liquids and water-based liquids;
a scent portion providing a smell component; and
a flavor portion providing a taste component, wherein the flavor portion is stevia.

18. The flavored perfume of claim 17, wherein the flavored perfume is a solid.

19. The flavored perfume of claim 17, wherein the flavored perfume is a liquid.

20. The flavored perfume of claim 17, wherein the flavored perfume is applied to a user's body in a manner that allows the user and others to both smell the flavored perfume with their nose and taste the flavored perfume with their tongue, wherein the flavored perfume has a product, and where the product is selected from the group consisting of is lipstick, balm, perfume, and roll-on perfume.
